(19) United States
(12) Patent Application Publication
(10) Pub. No.: US 2014/0315869 A1
(43) Pub. Date: Oct. 23, 2014

(54) THERAPEUTIC USES OF ECTOINE

(71) Applicant: BITOP AG, Witten (DE)

(72) Inventors: Klaus Unfried, Dusseldorf (DE); Ulrich Sydlik, Neuss (DE); Jean Krutmann, Wegberg (DE); Andreas Bilstein, Bergheim (DE)

(21) Appl. No.: 14/343,017

(22) PCT Filed: Sep. 7, 2012
(86) PCT No.: PCT/EP2012/003757

§ 371 Date: May 19, 2014

(30) Foreign Application Priority Data
Sep. 9, 2011 (DE) 10 2011 113 059.8

(51) Int. Cl. A61K 31/505; A61K 31/58

(52) U.S. Cl. CPC A61K 31/505; A61K 31/58
USPC 514/171; 544/335; 544/298

(57) ABSTRACT

The invention relates to a composition containing ectoine, hydroxyectoine and/or a salt, ester or amide of these compounds for the suppression of anti-apoptotic signals to neutrophil granulocytes and other cells taking part in inflammations. The delayed apoptosis of the neutrophils is a main component for various types of inflammation. Through the administration of ectoine restoring the normal rate of apoptosis is accomplished at least partially which is associated with a corresponding improvement of the inflammatory phenomena.

The invention relates to compositions containing ectoine, hydroxyectoine or associated salts, esters, and amides.

Osmolytes or compatible solutes from extremophilic microorganisms constitute a known group of low-molecular protective substances. Extremophiles are rather extraordinary microorganisms because they grow optimally and at high salt concentrations (up to 200 g NaCl/l) and elevated temperatures (60-110° C.) that in the event of mesophilic (normal) organisms would lead to an extensive damage of cellular structures. In recent years comprehensive research efforts have been made to identify the biochemical components that bring about the remarkable stabilization of the cell structures. Although many enzymes from hyperthermophilic microorganisms are stable even under elevated temperatures this cannot be generally said of the cellular structures of thermophilic and hyperthermophilic organisms. The high temperature stability of cell structures is—to a remarkable extent—due to low-molecular organic substances (compatible solutes, osmolytes) present in the intracellular environment. In recent years, various novel osmolytes could be identified in extremophilic microorganisms for the first time. In some cases it could be clearly shown that these compounds effectively contributed to the protection of cellular structures—first of all enzymes—against heat and dryness (K. Lippert, E. A. Galinski, Appl. Microbiol. Biotech. 1992, 37, 61-65; P. Louis, H. G. Trüper, E. A. Galinski, Appl. Microbiol. Biotech. 1994, 41, 684-688; Ramos et al., Appl. Environm. Microbiol. 1997, 63, 4020-4025; Da Costa, Santos, Galinski, Adv. in Biochemical Engineering Biotechnology, 61, 117-153).

For a number of compatible solutes useful application opportunities have been opened up in the medical, cosmetic, and biological field. Ectoine (2-methyl-1,4,5,6-tetrahydropyrimidine-4-carbocylic acid) and its derivatives count among the most important solutes. In publication EP 0 887 418 A2, for example, the use of ectoine and hydroxyectoine (5-Hydroxy-2-methyl-1,4,5,6-tetrahydropyrimin-4-carboxylic acid) is described for the treatment of skin diseases or as an effective addition for the cryoprotection of biological active agents and cells. DE 10 2006 056 766 A1 provides information about the use of ectoine for the treatment of the vascular leak syndrome (VLS). Further examples are the stabilization of vaccines (DE 100 65 986 A1) or the dermatological use for the treatment of neurodermatitis (DE 103 30 243 A1).

The structure of natural L-ectoine ((S)-2-methyl-1,4,5,6-tetrahydropyrimidine-4-carboxylic acid) is shown below:

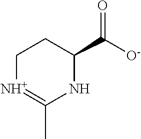

Also hydroxyectoine has been described as advantageous for various purposes. The structure of natural hydroxyectoine ((4S,5S)-5-hydroxy-2-methyl-1,4,5,6-tetrahydropyrimidine-4-carboxylic acid) is indicated hereunder:

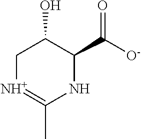

The treatment of pulmonary diseases due to the influence of airborne particulate matter and cardiovascular diseases is the subject of European pant EP 1 641 442 B1. It describes the inhalation of pharmaceutical preparations containing ectoine or hydroxyectoine for combating such diseases. However, diseases that are not attributable to airborne particulates are not subject matter of said patent.

In numerous inflammatory phenomena neutrophil granulocytes, for short neutrophils, play an important role, especially for combating viral and bacterial pathogens. Neutrophils are formed in large quantities in bone marrow. The pathogens are destroyed by the liberation of reactive oxygen species and enzymes such as myeloperoxidase, elastase or matrixmetalloproteinases. However, since these reactions have side effects on the relevant tissue a strict regulation must take place to ensure the neutrophilic inflammation does not last longer than is necessary for the combating of the actual pathogens. Accordingly, a signaling cascade is activated that leads to apoptosis of the neutrophil granulocytes. However, inflammatory mediators ensure that the apoptosis is delayed and in this way cause the life span of the neutrophils to be prolonged. The accumulation of neutrophils and monocytes in the infected area constitutes one of the main components of an inflammation. A long-lasting delay of the apoptosis may even result in chronic inflammatory phenomena. Examples in this case are a chronic lung inflammation or a chronic obstructive pulmonary disease (COPD).

Therefore, with a view to combating chronic inflammations emphasis must be on controlling inflammations that are caused by an accumulation of neutrophils. Problems in this context are encountered in that neutrophils unlike other cells participating in an inflammation only respond poorly to corticosteroids. For that reason, medical substances would be desirable that inhibit the anti-apoptotic effect of inflammatory mediators, corticosteroids, and other substances.

Surprisingly, it has now been found that treatment with ectoine or hydroxyectoine eliminates at least partially the anti-apoptotic effect of inflammatory mediators, corticosteroids, and other substances and in this manner restores the natural apoptosis rate of neutrophil granulocytes, however, without having a proapoptotic effect by itself. The invention, therefore, relates to a composition containing ectoine, hydroxyectoine and/or a salt, ester or amide of these compounds for the suppression of anti-apoptotic signals to neutrophil granulocytes and other cells taking part in inflammations such as macrophages, eosinophil granulocytes, basophil granulocytes, mast cells, lymphocytes, epithelioid cells, and dendritic cells. The suppression of the anti-apoptotic signals is normally aimed at in the context of treating or preventing inflammations, with chronic inflammations playing a special role here. Of special significance is the treatment of inflammations affecting the respiratory tract and the lung, in particular pneumonia, asthma, chronic obstructive pulmonary disease (COPD), ARDS, cystic fibrosis, pulmonary fibrosis, silicosis, sarcoidosis, allergies, and bronchial hyper-responsiveness.

In conjunction with the inflammatory reactions examined the inhibition of the apoptosis of neutrophil granulocytes is expected to be caused by a membrane-mediated activation of membrane-coupled signaling pathways via PI3-K (phosphatidylinositol-3-kinase). These lead to an activation of protein kinase B (AKT) and ultimately to an increase of the Mcl-1 level, an anti-apoptotically acting protein. It is assumed that ectoine diminishes the AKT activation.

Neutrophil inflammation reactions were examined in rats with carbon nanoparticles intratracheally administered. This involved administration both with and without ectoine. The rats were subsequently examined at different times, with a significant reduction of the amount of neutrophils being observed after two days in the ectoine group in comparison to the placebo group. The effectiveness observed after two days coincides with the detected reduction of the liberation of cinc-1, a chemokine playing an important role in inflammatory phenomena. In the first place, the liberation of cinc-1 is initially due to epithelial cells and macrophages, whereas later on the liberation caused by the great amount of ultimately existing neutrophils dominates. With ectoine administered, the reduction of liberated cinc-1 after two days shows that the number of neutrophils could be brought down at that time.

It could also be demonstrated that an administration of ectoine in two doses one and two days after commencement of the inflammatory reaction virtually had the same effect as an ectoine administration at the time the inflammatory reaction was triggered. It thus follows that ectoine cannot only be used on a preemptive basis but also for the treatment of an already existing inflammation. A diminishing of the amount of neutrophils as well as a lowering of the cinc-1 level were also observed when ectoine had been administered repeatedly after an inflammation reaction had been triggered off several times, which even underlines its usefulness for the treatment of chronic inflammatory phenomena.

Corresponding investigations were also carried out with isolated human neutrophil granulocytes. It could be demonstrated that a reduction of the apoptosis rate due to pro-inflammatory factors such as carbon nanoparticles (CNP), LTB₄ or GM-CSF can be compensated depending on concentration at least partially by administering ectoine. Solely applying ectoine to neutrophils without a previous treatment with pro-inflammatory factors did not result in an increase of the apoptosis rate. It is thus evident that ectoine has no pro-apoptotic effect basically but rather causes the anti-apoptotic mechanisms involved in inflammatory phenomena to be suppressed.

Although the anti-anti-apoptotic effectiveness had been proved by in vivo and in vitro experiments during which an inflammatory reaction was triggered with the help of carbon nanoparticles said effectiveness is by no means, however, limited in this respect but the present invention rather relates explicitly also to such inflammations that are not attributable to the influence of airborne particulate matter. Whereas in EP 1 641 442 B1 it was previously proposed that ectoine only counteracted the detrimental effects of airborne particulate matter directly it has now been demonstrated that the treatment of inflammations with ectoine begins with and involves restoring the natural apoptosis rate of neutrophils.

Moreover, a combination of ectoine/hydroxyectoine resp. relevant derivatives with corticosteroids has proved to be particularly beneficial, in particular with glucocorticoids such as dexamethasone, budesonide, betamethasone, triamcinolone, fluocortolone, methylprednisolone, deflazacort, prednisolone, prednisone, cloprednole, cortisone, hydrocortisone, fluocortine, clocortolone, clobetasone, alclomethasone, flumethasone, fluoprednidene, fluorandrenolone, prednicarbate, mometasone, methylprednisolone, fluticasone, halometasone, fluocinolone, diflorasone, desoximetasone, fluocinonide, fludrocortisone, deflazacort, rimexolone, cloprednole, amcinonide, halcinonide, diflucortolone, clobetasol or salts, esters, amides, solvates or hydrates of these compounds.

Although corticosteroids are known as active agents counteracting inflammatory phenomena their role in combating neutrophil inflammations is equivocal since they cause the natural neutrophil apoptosis to diminish. Accordingly, by bringing a corticosteroid to act in conjunction with ectoine/hydroxyectoine the advantageous anti-inflammatory effect of the steroid is combined with the effect of ectoine/hydroxyectoine restoring the natural apoptosis rate and thus reducing the undesirable anti-apoptotic effect of the corticosteroid. Examples are combining ectoine or relevant derivatives with dexamethasone and/or budesonide. Other favorable alternative combinations are ectoine/hydroxyectoine used in conjunction with GM-CSF, leukotrienes such as LTB₄, theophylline (1,3-dimethylxanthine), leukotriene antagonists, phosphodiesterase inhibitors (PDE inhibitors, in particular PDE4 inhibitors), muscarinic receptor antagonists, anti-cholinergic agents such as ipratropium bromide or tiotropium bromide or other pharmaceutical substances causing the natural neutrophil apoptosis rate to be undesirably reduced.

In the context of combining corticosteroids with ectoine/hydroxyectoine considerable significance must also be attached to the treatment of lung diseases, in particular pneumonia, asthma (bronchial asthma), chronic obstructive pulmonary disease (COPD), ARDS, cystic fibrosis, pulmonary fibrosis, silicosis, sarcoidosis, allergies, and bronchial hyperresponsiveness. It is considered expedient in this case to provide the composition in the form of an inhalable composition. For this purpose, the composition may be provided in liquid form as a solution or in solid form, with said composition being atomized as aerosol and inhaled, if necessary and expedient with the help of an inhalation device.

The administration of corticosteroids and ectoine/hydroxyectoine must not necessarily take place using them in the same composition; it is important, however, that they are administered simultaneously or within a narrow time frame so that the active substances can jointly take effect functionally in the way described hereinbefore. Accordingly, the invention also relates to a combination preparation comprising at least two individual compositions, that is to say one composition containing ectoine, hydroxyectoine and/or a salt, ester or amide of these compounds as well as an additional composition containing a corticosteroid. The combination preparation thus constitutes a kit consisting of parts of two compositions which can only be fully effective when applied together. The corticosteroid may in particular be one of the glucocorticoids referred to hereinbefore.

Pharmacologically compatible salts of the ectoine/hydroxyectoine embrace alkaline or alkaline-earth salts, in particular the salts of potassium, sodium, magnesium and calcium but also salts with organic bases such as, for example, with nontoxic aliphatic or aromatic amines.

Through the reaction of the carboxyl group of the ectoine/hydroxyectoine with alcohols or amines relevant esters or amides can be obtained which may also be employed within the scope of the invention. In the event of an amide the nitrogen atom may in turn comprise saturated or unsaturated, straight-chained or branched alkyl groups. In case of hydroxyectoine also the hydroxy group may be subjected to a reaction with a carboxylic acid to form a relevant ester.

It has turned out that, inter alia, the use of ectoine amide of 2-hydroxy-5-aminobenzoic acid offers advantages. The structural formula is as follows:

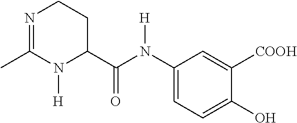 [SMILES: CC1=NCCC(C(=O)Nc2ccc(O)c(C(=O)O)c2)N1]

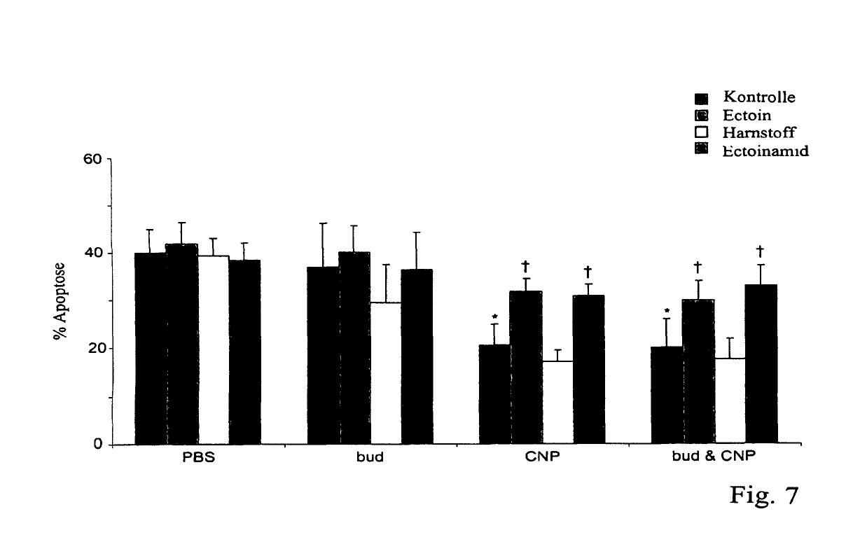

Therefore, this is 2-methyl-1,4,5,6-tetrahydropyrimidine-4-carboxylic acid amide of 2-hydroxy-5-aminobenzoic acid. Preferably, it is the relevant amide of the L-ectoine: (S)-2-methyl-1,4,5,6-tetrahydropyrimidine-4-carboxylic acid amide. The compound was tested and showed effectiveness comparable to ectoine itself (cf. FIG. 7). It is thus also possible to use the respective amides of hydroxyectoine (5-hydroxy-2-methyl-1,4,5,6-tetrahydropyrimin-4-carboxylic acid), preferably of L-hydroxyectoine ((4S,5S)-5-hydroxy-2-methyl-1,4,5,6-tetrahydropyrimidine-4-carboxylic acid), i.e. the hydroxyectoine amide of 2-hydroxy-5-aminobenzoic acid. The relevant amide as well may be present in ionic or zwitterionic form. The invention thus relates also to the mentioned compounds, respectively salts, esters or amides of these compounds and compositions that contain these compounds, respectively salts, esters or amides. The compositions can be put to use as pharmaceutical preparations, in particular for the suppression of anti-apoptotic signals acting on neutrophil granulocytes, macrophages, eosinophil granulocytes, basophil granulocytes, mast cells, lymphocytes, epithelioid cells, dendritic cells or other cells participating in inflammations.

Generally speaking, the inventive active agents if thought expedient with further active agents may be processed to obtain preferably inhalable medicaments making use of auxiliary substances and additives pharmacologically unobjectionable. In the event of inhalable liquid preparations such additives primarily consist of water to which, as the case may be, further solvents, stabilizers, preservation agents, emulsifiers, antioxidants, fillers or solutizers are added. As further active agents antiasthmatics, broncholytics, non-steroidal anti-inflammatory drugs (NSAIDs) or expectorants are conceivable. Appropriate preservation agents are: benzalkonium chloride, chlorobutanol, thiomersal, methyl paraben, propyl paraben, sorbic acid and salts thereof, sodium edetate, phenylethyl alcohol, chlorhexidine hydrochloride acetate, -digluconate, cetylpyridinium chloride, -bromide, chlorocresol, phenylmercuric acetate, phenylmercuric nitrate, phenylmercuric borate, phenoxyethanol.

The formulations proposed by the invention may also contain suitable buffer systems or other auxiliary substances for pH adjustment to adjust and maintain a pH value in the range of between 4 and 8, preferably between 5 and 7.5. Suitable buffer systems are citrate, phosphate, trometamol, glycin, borate, acetate. These buffer systems may be produced from substances such as citric acid, monosodium phosphate, disodium phosphate, glycin, boric acid, sodium tetraborate, acetic acid or sodium acetate.

Typically, the concentration of ectoine/hydroxyectoine resp. the relevant derivative ranges between 0.001 and 50% w/w, preferably 0.05 and 20% w/w, in particular between 0.1 and 10% w/w based on the composition.

In the event of an administration in solid form, for example by means of powder inhalers it is recommendable that only carrier substances are used that are easily resorbed and non-irritating such as micronized lactose.

### EXPERIMENT 1

Carbon nanoparticles (CNP, 14 nm, Printex 90, Degussa, Frankfurt, Germany) were suspended in a phosphate buffered salt solution (PBS) by means of ultra-sonic treatment. Similarly, a 0.1 and a 1 mM solution of ectoine was produced in PBS. 0.4 ml of the CNP particle suspension was administered intratracheally to Fisher 344 female rats. Treatment with 0.4 ml of the ectoine solutions, respectively PBS took place after one and two days. The rats were sacrificed on the third day, their lungs purged with 4×5 ml of PBS each. The cells of the individual animals were suspended in 1 ml of PBS and centrifuged. The pellets were washed with PBS once and resuspended in 300 μl of hypotonic solution (0.1% sodium citrate, 0.1% triton×100) containing 50 μg/ml of propidium iodide (PI). To determine the rate of apoptosis a fluorescence measurement was finally performed.

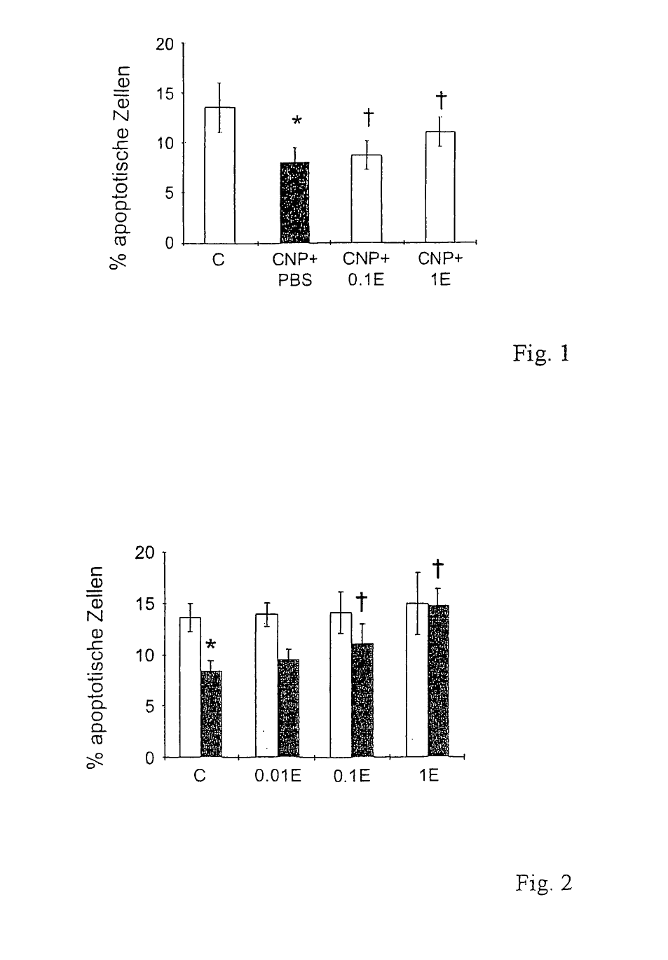

The results obtained are illustrated in FIG. 1 (C: control without CNP treatment; *: significant difference with respect to control group without CNP treatment; †: significant difference with respect to animals treated only with CNP and PBS).

### EXPERIMENT 2

The effect of ectoine on the apoptosis was investigated by way of human neutrophils. Neutrophils from young, healthy donors (3 male and 2 female) were isolated and treated with the amounts of ectoine (mM) indicated. Treatment was carried out with ectoine alone (open columns) and with 33 μg/ml of CNP (black columns). Ectoine was not administered in the control group (C). The results are shown in FIG. 2.

The apoptotic cells were quantified in the following manner: The neutrophils were suspended in 300 μl of hypotonic solution containing propidium iodide (PI). The fluorescence of PI was determined by means of flow cytometry (FACScan cytometer, BD Biosciences). The results are shown as percentage of hypodiploidal DNA (sub-G1), corresponding to fragmented DNA which is characteristic of apoptotic cells.

The reduction of the apoptosis rate caused by CNP could virtually be eliminated by administering significant amounts of ectoine.

(*: significant difference with respect to control group without CNP treatment; †: significant difference with respect to neutrophils treated with CNP and PBS only).

### EXPERIMENT 3

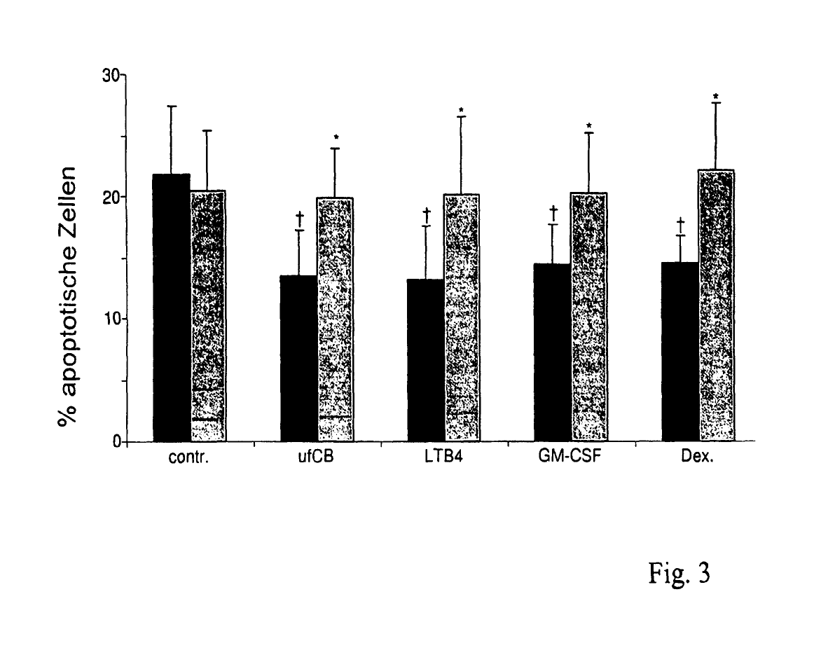

The effect of ectoine on the apoptosis was investigated by way of human neutrophils. Neutrophils of young, healthy donors (3 male and 2 female) were isolated and pretreated for 2 hours with PBS (dark columns) and with 1 mM of ectoine (light columns). Subsequently, treatment took place with 33 μg/ml of carbon nanoparticles (ufCB: ultrafine carbon black), 300 nM LTB₄, 20 ng/ml GM CSF or 1 μM of dexamethasone, respectively no treatment with proinflammatory factors. The results are shown in FIG. 3. (†: significant difference with respect to control group; *: significant difference with respect to neutrophils treated with proinflammatory factors and PBS only; quantification of the apoptotic cells analogous to Experiment 2).

### EXPERIMENT 4

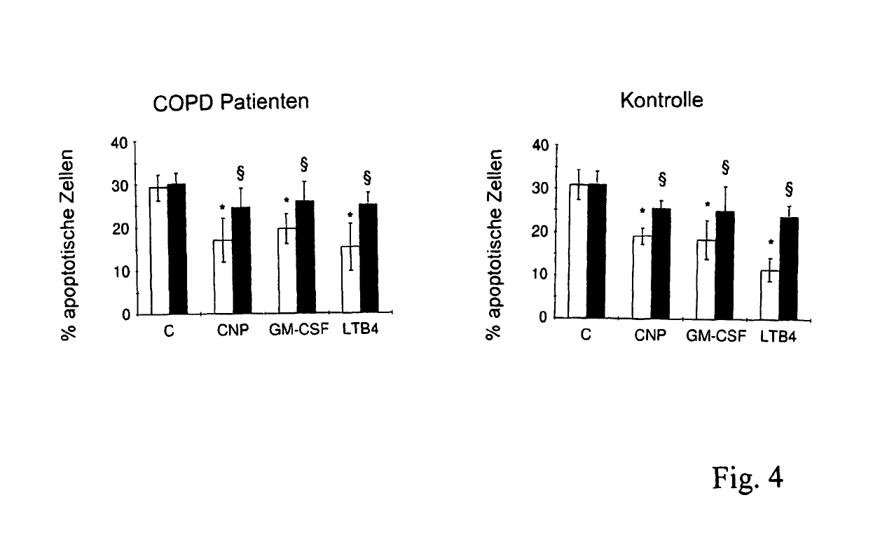

The effectiveness of ectoine on apoptosis was demonstrated analogously to Experiment 3 with COPD patients and non-COPD patients of corresponding age. The neutrophil granulocytes were pretreated for 2 hours with 1 mM of ectoine resp. PBS following which treatment took place for 16 hours with 33 μg/ml CNP, 300 nM LTB₄, 20 ng/ml GMCSF or PBS. While a higher base apoptosis was noted the apoptosis was nevertheless reduced by the effect of inflammatory stimulants, and an additional treatment with ectoine resulted in the apoptosis rate to be restored significantly. The results are shown in FIG. 4. (dark columns: pretreatment with ectoine; light columns: pretreatment with PBS; *: significant difference with respect to control group without CNP or inflammation mediators; §: significant difference with respect to the treatment without ectoine; quantification of the apoptotic cells analogously to Experiment 2).

### EXPERIMENTS 5-7

For Experiments 5 to 7 neutrophil granulocytes were obtained from blood samples. Groups 1 and 2 consisted of persons who participated in a current patient study. Male patients (aged 40 to 80 years) with stable COPD history (GOLD III/IV) and healthy control patients from an identical age group.

In addition, young male volunteers were found in the clinic (group 3).

### EXPERIMENT 5

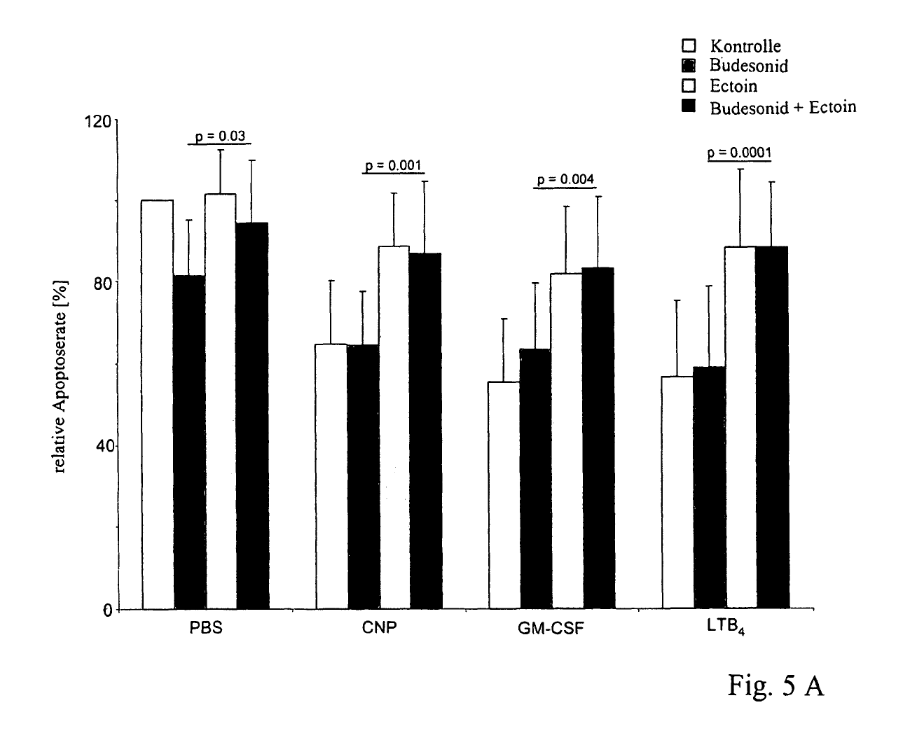

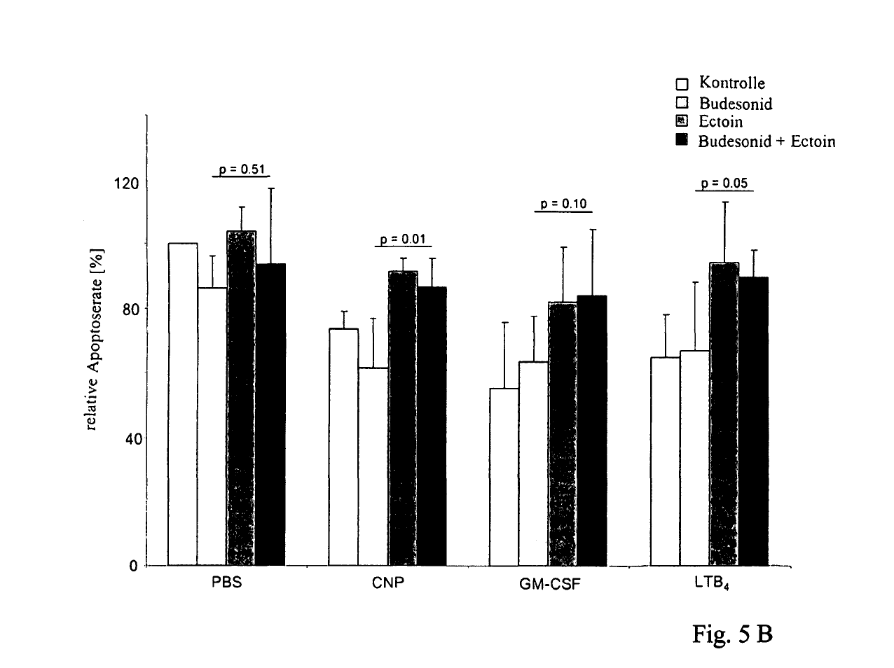

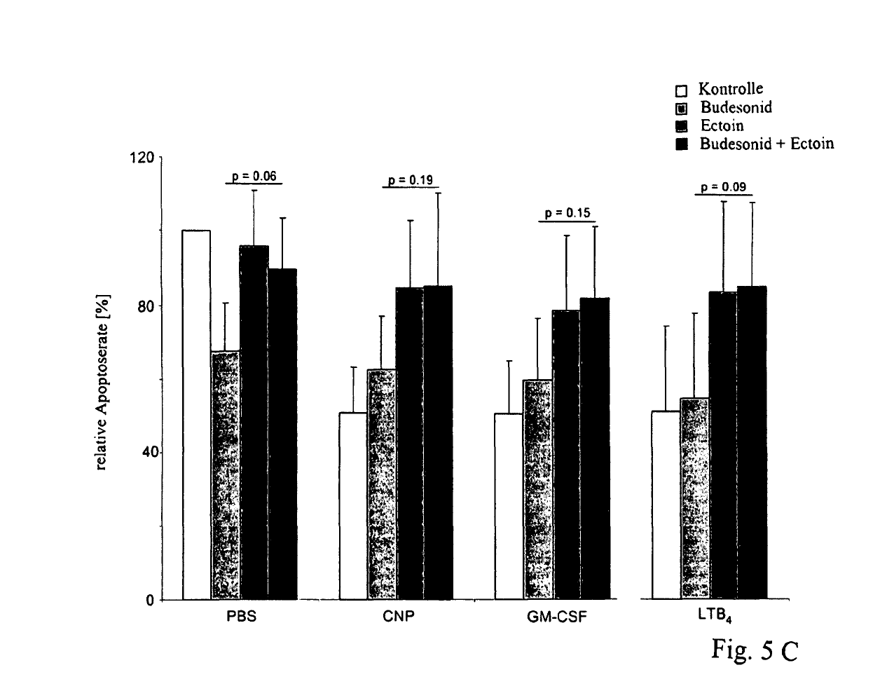

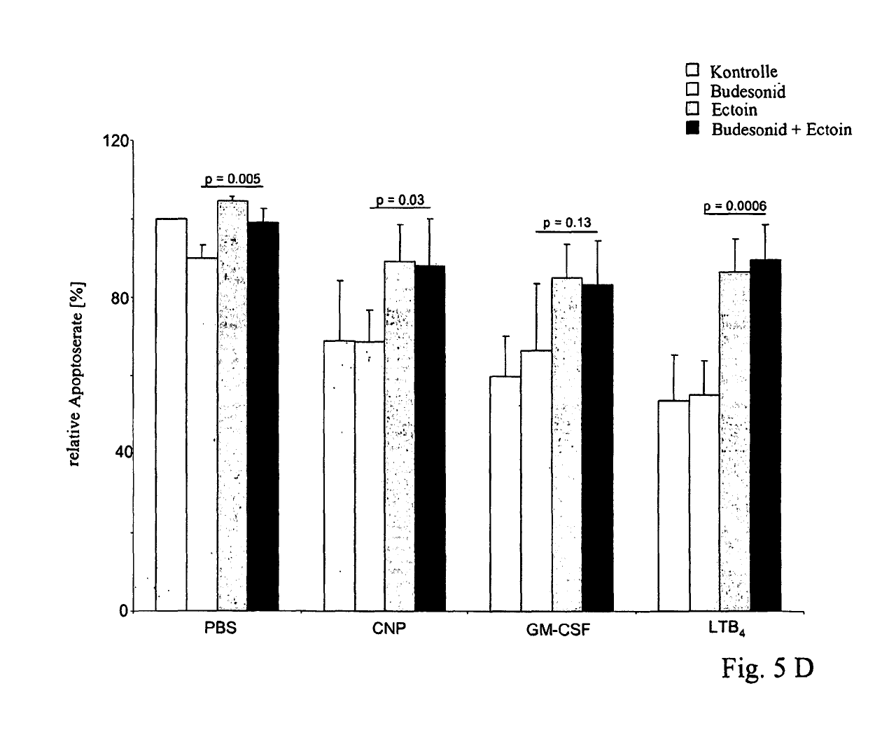

The influence of ectoine in combination with the corticosteroid budesonide on the apoptosis rates of neutrophils is illustrated in FIG. 5. Sub-G₁-cells were measured after propidium iodide coloration (FACS-flow cytometry). Cells: primary, peripheral neutrophils. Isolation of the neutrophils by Percoll® centrifugation. Cultivation of 2×10⁶ neutrophils in the presence of 33 μg/ml CNP, 300 nM LTB₄, 20 ng/ml GM-CSF, 1 μM budesonide, 1 mM ectoine and combinations thereof for 16 h. FIG. 5 A: cells cumulated from all samples (n=15), FIG. 5 B: cells from COPD patients (n=5), FIG. 5 C: cells from healthy age control group (n=5), FIG. 5 D: cells from young volunteers (n=5).

Treatment with budesonide leads to a decrease of the apoptosis rate of neutrophils. It can be seen that a pretreatment of the cells with 1 mM of ectoine obviates the anti-apoptotic effect of the budesonide significantly. The effect occurred in any one of the groups and also in the groups as a whole. The effect could be observed with neutrophil granulocytes that were not treated with proinflammatorily acting substances. However, when proinflammatorily acting substances (CNP, LTB₄, GM-CSF) were combined with budesonide the anti-apoptotic effect was successfully prevented by ectoine as well.

### EXPERIMENT 6

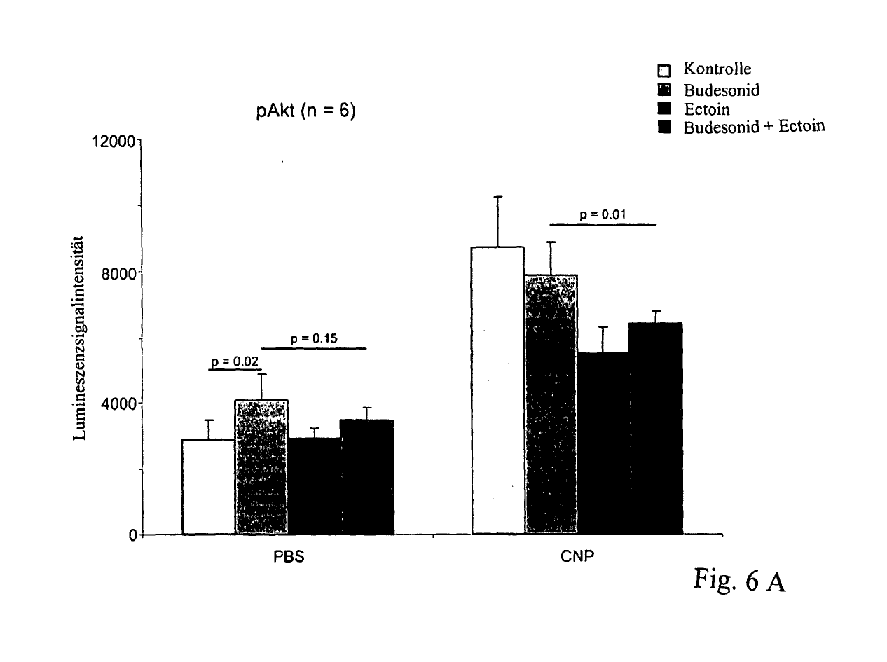

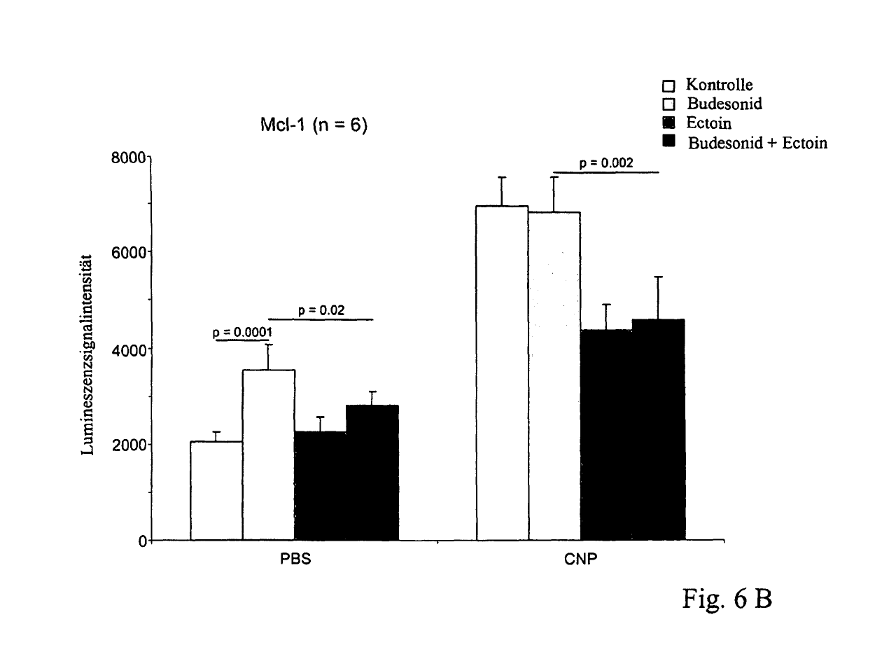

The influence of ectoine in combination with budenoside on anti-apoptotic signals can be seen from FIG. 6. With selected samples of neutrophils relevant signals were detected via protein kinase B (Akt) and Mcl-1 by measurement of the Akt phosphorylation and the Mcl-1 protein level, with human primary peripheral neutrophils being used as cells. Isolation of the neutrophils by Percoll® centrifugation, the cultivation of 2×10⁶ neutrophils in the presence of 33 μg/ml CNP, 1 μM budesonide, 1 mM ectoine and combinations thereof for 6 h. A protein isolation, western blot, luminescence on x-ray films was effected of material from each 3 COPD patients and 3 persons from the corresponding age control group. From the results the anti-apoptotic effect of budesonide can be clearly seen that activates the Akt signaling pathway and increases the amount of anti-apoptotic protein Mcl-1. Ectoine is capable of counteracting this effect, also in the presence of CNP.

### EXPERIMENT 7

The influence of various other test substances on the apoptosis rate of human neutrophils is shown in FIG. 7. Sub-G1-cells were measured after propidium iodide coloration (FACS-flow cytometry). Cells: primary, peripheral, human neutrophils. Isolation of the neutrophils by Percoll® centrifugation and cultivation of 2×10⁶ neutrophils in the presence of 33 μg/ml CNP, 1 μM budesonide, 1 mM ectoine, 1 mM urea, 1 mM ectoine amide and combinations thereof for 16 h. The cells were obtained from young volunteers (group 3), n=5, bud=budenoside. Two additional test substances (urea and the ectoine amide of 2-hydroxy-5-aminobenzoic acid) were examined to check whether they were capable of inhibiting anti-apoptotic effects on neutrophils. Both substances did not have an influence on the background apoptosis rate. Ectoine amide was found to be able to prevent the reduction of the apoptosis rate that was caused by carbon nano-particles (CNP) with or without budenoside, whereas this could not be achieved with urea.

1. Composition containing ectoine, hydroxyectoine and/or a salt, ester or amide of these compounds for the suppression of anti-apoptotic signals to neutrophil granulocytes, macrophages, eosinophil granulocytes, basophil granulocytes, mast cells, lymphocytes, epithelioid cells, dendritic cells or other cells participating in inflammations.

2. Composition according to claim 1, characterized in that the suppression of the anti-apoptotic signals takes place in the treatment or prevention of an inflammation.

3. Composition according to claim 2, characterized in that the inflammation is a chronic inflammation.

4. Composition according to claim 2, characterized in that the inflammation concerns pneumonia, asthma, a chronic obstructive pulmonary disease, ARDS, cystic fibrosis, pulmonary fibrosis, silicosis, sarcoidosis, allergy, or bronchial hyperresponsiveness.

5. Composition according to claim 1 characterized in that the composition contains at least a corticosteroid.

6. Composition according to claim 5, characterized in that the corticosteroid is a glucocorticoid.

7. Composition according to claim 6, characterized in that the glucocorticoid is dexamethasone, budesonide, betamethasone, triamcinolone, fluocortolone, methylprednisolone, deflazacort, prednisolone, prednisone, cloprednole, cortisone, hydrocortisone, fluocortine, clocortolone, clobetasone, alclomethasone, flumethasone, fluoprednidene, fluorandrenolone, prednicarbate, mometasone, methylprednisolone, fluticasone, halometasone, fluocinolone, diflorasone, desoximetasone, fluocinonide, fludrocortisone, deflazacort, rimexolone, cloprednole, amcinonide, halcinonide, diflucortolone, clobetasol or a salt, ester, amide, solvate or hydrate of one of the above mentioned compounds.

8. Composition containing ectoine, hydroxyectoine and/or a salt, ester or amide of these compounds for the treatment or prevention of pulmonary diseases except those attributable to the influence of airborne particulate matter.

9. Composition containing ectoine, hydroxyectoine and/or a salt, ester or amide of these compounds as well as a corticosteroid for application in the prevention and/or treatment of pulmonary diseases.

10. Composition according to claim 8, characterized in that the pulmonary disease is pneumonia, a chronic obstructive pulmonary disease, asthma, ARDS, cystic fibrosis, pulmonary fibrosis, silicosis, sarcoidosis, allergy, or bronchial hyperresponsiveness.

11. Composition according to claim 1 characterized in that the composition is an inhalable composition.

12. Combination preparation of compositions for the application in the prevention and/or treatment of pulmonary diseases, wherein the compositions are provided for administration within a narrow time frame, consisting of at least
a first composition containing ectoine, hydroxyectoine and/or a salt, ester or amide of these compounds and
a second composition containing a corticosteroid.

13. Compound of the structural formula 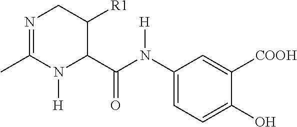
or a salt, ester or amide of this compound, with R1=H, OH or OR2 with R232 alkyl, cycloalkyl or aryl, preferably C₁ to C₁₀ alkyl, C₁ to C₁₀ cycloalkyl or C₁ to C₁₀ aryl.

14. Composition containing a compound and/or a salt, ester or amide according to claim 13 for the use as pharmaceutical agent.

15. Composition containing a compound and/or a salt, ester or amide according to claim 13 for the suppression of anti-apoptotic signals to neutrophil granulocytes, macrophages, eosinophil granulocytes, basophil granulocytes, mast cells, lymphocytes, epithelioid cells, dendritic cells or other cells participating in inflammations.
